(19) United States
(12) Patent Application Publication
(10) Pub. No.: US 2005/0283146 A1
(43) Pub. Date: Dec. 22, 2005

(54) Thermally extended spiral cryotip for a cryoablation catheter

(76) Inventor: David J. Lentz, La Jolla, CA (US)

Correspondence Address: NEIL K. NYDEGGER;NYDEGGER & ASSOCIATES, 348 Olive Street, San Diego, CA, 92103, US

(21) Appl. No.: 10/869,991

(22) Filed: Jun. 17, 2004

(51) Int. Cl. A61B 18/02

(52) U.S. Cl. 606/21; 606/23

(57) ABSTRACT

A system and method for cryoablating tissue at a target site in a patient includes a cryotip attached to the distal end of a catheter tube. The cryotip is made of a shape memory material that assumes a straight configuration at a first temperature and a coiled configuration at a second temperature. With the cryotip in the straight configuration, the cryotip is guided through the vasculature of a patient to the target site. A refrigerant fluid is introduced into the expansion chamber of the cryotip to cool the cryotip to the second temperature. At the second temperature, the cryotip transforms into the coiled configuration and is placed in contact with circumferential tissue around the target site. The circumferential tissue is then cryoablated in a single-step operation.

### FIELD OF THE INVENTION

The present invention pertains generally to systems and methods for cryoablating tissue in the vasculature of a patient. More particularly, the present invention pertains to systems and methods for cryoablating a circumferential band of tissue. The present invention is particularly, but not exclusively, useful as a single step process to treat atrial fibrillation by creating a substantially circumferential lesion around an ostium of a pulmonary vein where the pulmonary vein connects with the left atrium.

### BACKGROUND OF THE INVENTION

Atrial fibrillation is the most common form of heart arrhythmia. In a normally functioning heart, an electrical system directs electrical impulses through the heart in an organized fashion to stimulate the heart so that it properly contracts. Specifically, the upper chambers (atria) and the lower chambers (ventricles) of the heart are stimulated to contract in a synchronous manner. Fundamentally, atrial fibrillation is the loss of synchronicity between the upper chambers and the lower chambers of the heart. In effect, atrial fibrillation is a very fast, uncontrolled heart rhythm in which the atria quiver instead of beating. Atrial fibrillation can also be described as a storm of electrical energy that travels across both atria causing them to fibrillate at 300 to 600 times a minute. This storm of electrical energy interferes with the electrical system of the heart and prevents the heart from functioning properly.

Research has shown that almost all atrial fibrillation is due to abnormal electrical signals that pass through (or originate at) the tissue surrounding the ostia of the pulmonary veins where the pulmonary veins connect with the left atrium. Inside the heart, these abnormal electrical signals can disrupt the electrical system and cause the heart to beat abnormally. Accordingly, preventing the abnormal electrical signals from reaching the heart is one method for treating atrial fibrillation. In one such treatment method, a circumferential band of tissue surrounding the ostium of a pulmonary vein is ablated to destroy tissue and create a conduction block. Once ablated, the destroyed tissue is no longer able to initiate or conduct any type of electrical signal. Accordingly, abnormal electrical signals from the pulmonary vein are prevented from reaching the heart.

One technique for ablating the tissue surrounding the ostium of a pulmonary vein involves cryoablating the tissue with a cryoablation catheter. To date, this technique has typically required a plurality of locations to be sequentially ablated. To achieve this, the cold cryotip of the cryoablation catheter must be repeatedly moved (i.e. reoriented) to sequentially contact portions of tissue around the periphery of the ostium. In theory, these ablations can combine to establish an effective circumferential ablation band. However, in practice, this complex process often results in a non-uniform or discontinuous circumferential lesion that does not adequately block all of the abnormal electrical signals from entering the heart. Moreover, this procedure is time consuming (increasing patient risk) because it requires extensive manipulation of the cryotip around the ostium.

The present invention contemplates the cryoablation of a circumferential band of tissue in a single-step (i.e. the entire band of tissue is ablated simultaneously). This requires contacting the circumferential band of tissue with a contacting element having a relatively large-diameter, somewhat cylindrical shaped contact surface. The problem, however, has been the non-invasive delivery of a contacting element having this relatively large, bulky shape to the treatment site. In particular, the human vasculature is curved, branched and contains vessels having relatively small inner diameters. As a consequence, it is necessary to design a catheter having a relatively low profile to allow the distal end of the catheter to navigate through the complex vasculature. To solve this dilemma, the present invention contemplates a contacting element that can be reshaped in-situ from a relatively low profile shape to a shape suitable for contacting a circumferential band of tissue.

With the above in mind, certain alloys, called shape-memory alloys, are known for their ability to recover relatively large strains. As is well known, the crystal structure of alloys can be manipulated by thermal treatments and other processes to alter the alloy's microstructure from one crystal structure to another. Each crystal structure is known as a phase, such as an austenite phase or a martensite phase, and the change from one phase to another is termed a phase transformation. To use a traditional, one-way shape-memory alloy, a part is initially shaped from the alloy at a first temperature, above the phase transformation temperature. Next, the shaped part can be cooled to a second temperature, below the phase transformation temperature, thus inducing a phase transformation such as an austenite to martensite phase transformation. At the lower temperature, while the alloy is still in the martensite phase, a stress can be applied to deform the part to strains of up to approximately 8 percent. Upon release of the applied stress, the 8 percent strain will remain. Next, the deformed part can be heated back above the phase transformation temperature, thereby transforming the alloy back to the austenite phase. During this last phase transformation, the strain will be recovered, and the original (unstrained) shape of the part will return.

More recently, two-way shape memory alloys have been developed. These alloys have the ability to recover a first preset shape when cooled below their transformation temperature and return to a second preset shape when subsequently heated above their transformation temperature. These shapes can be preset, for example, using a training process that includes an overdeformation while the alloy is in the martensitic phase. Alternatively, a cool-deform-heat cycle can be performed two or more times to preset the shapes.

In light of the above, it is an object of the present invention to provide a system and method for performing a non-invasive, single-step cryoablation of a circumferential shaped band of tissue in the vasculature of a patient. Another object of the present invention is to provide a system and method for treating atrial fibrillation by cryoablating the peripheral tissue around the ostium of a pulmonary vein where the pulmonary vein connects to the left atrium. Still another object of the present invention is to provide a system and method for cryoablating tissue in the vasculature of a patient in an efficient and reliable manner.

### SUMMARY OF THE INVENTION

In accordance with the present invention, a system and method are provided for cryoablating tissue in the vasculature of a patient. In one particular application of the present invention, the peripheral tissue surrounding the ostium of a pulmonary vein where the pulmonary vein connects to the left atrium is ablated in a single step, relatively non-invasive process to treat atrial fibrillation.

For the present invention, the system includes a cryotip that is attached to the distal end of a catheter tube. More specifically, the cryotip can include a contact segment and a cryo-element. In greater detail, the contact segment is attached to the cryo-element to establish a thermally conductive interface therebetween. With this cooperation of structure, the cryo-element can be cooled to lower the temperature of the contact segment.

Structurally, the contact segment is transformable in response to a temperature decrease from a first, relatively straight configuration to a second, substantially curved (e.g. coiled or spiral) configuration. In addition, the contact segment is transformable from the coiled configuration back to the relatively straight configuration in response to a temperature increase. In the relatively straight configuration, the contact segment can be somewhat easily passed through the vasculature to (and from) a treatment site. On the other hand, when the contact segment is in the coiled configuration, the contact segment can be cooled to a cryogenic temperature to cryoablate a circumferential band of tissue in a one-step process.

For the cryoablation system, the contact segment is made of a thermally-conductive, shape memory material that has been formed having a preset, relatively straight shape at a first, relatively high temperature (T₁), and having a preset, substantially coiled shape at a second, relatively low temperature (T₂). In the coiled configuration, the contact segment has a sufficiently large coil diameter to establish contact with the circumferential band of tissue that is to be cryoablated.

To cool the contact segment, the cryo-element is formed with an expansion chamber. In one embodiment, the cryoablation system includes a supply tube that is positioned inside the lumen of the catheter tube. In one implementation, the supply tube is positioned inside the lumen of the catheter tube to establish a return line between the inner surface of the catheter tube and the outer surface of the supply tube.

The system can further include a refrigerant supply unit that is positioned at an extracorporeal location to introduce a fluid refrigerant into the proximal end of the supply tube. The fluid refrigerant then traverses through the lumen of the supply tube and exits the supply tube into the expansion chamber of the cryo-element. In one implementation, a flow restricting device, such as a capillary tube, can be used to restrict flow at the distal end of the supply tube. In this implementation, the fluid refrigerant passes through the restriction and then expands into the chamber to cool the cryo-element. In a particular embodiment of the present invention, a fluid refrigerant is used that transitions from a liquid state to a gaseous state as it expands into the cryo-element chamber. Heat absorbed by the refrigerant during this phase transition (i.e. latent heat) cools the cryo-element. After expansion, the gaseous fluid refrigerant can pass through the return line and exit the patient at the proximal end of the catheter tube.

In the operation of the cryoablation system, the cryotip is initially maintained at a temperature that is at or above the first temperature (T₁) to thereby configure the cryotip in a straight configuration. Typically, an alloy composition is used wherein the first temperature (T₁) is at or below ambient room temperature allowing the cryotip to be in the straight configuration at both room temperature and at body temperature. While the cryotip is at or above the first temperature (T₁), the cryotip is advanced through the vasculature of a patient to the treatment site.

Next, a refrigerant fluid (e.g. nitrous oxide) is passed through the supply tube for expansion into the expansion chamber of the cryo-element. This expansion cools the cryo-element, which in turn, cools the contact segment to the second temperature (T₂), transforming the cryotip into the coiled configuration and into contact with a circumferential band of target tissue. Next, the contact segment is maintained at the second temperature (T₂), which is typically approximately minus 85 degrees Celsius, until the target tissue is adequately cryoablated.

After the target tissue has been cryoablated, the cryotip is allowed to warm to a temperature that is at or above the first temperature (T₁) to configure the cryotip into the straight configuration. Once the cryotip has transformed into the straight configuration, the cryocatheter can be withdrawn from the vasculature of the patient.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a perspective view of a cryoablation system with peripheral components of the system shown schematically;

FIG. 2 is a cross-sectional view of a distal portion of the cryoablation system shown in FIG. 1, as seen along the line 2-2 in FIG. 1;

FIG. 3 is a perspective view of a distal portion of the cryoablation system shown in FIG. 1, shown in the straight configuration and positioned at a treatment site in the vasculature of a patient;

FIG. 4 is a perspective view of the a distal portion of the cryoablation system shown in FIG. 1, shown in the coiled configuration and positioned at a treatment site in the vasculature of a patient;

FIG. 5 is a cross-sectional view as in FIG. 2 illustrating an alternate embodiment of a cryoablation system having a cryo-element that is positioned proximal to the contact segment; and

FIG. 6 is a cross-sectional view as in FIGS. 2 and 3 illustrating an alternate embodiment of a cryoablation system having a contact segment that surrounds and establishes an expansion chamber.

### DESCRIPTION OF THE PREFERRED EMBODIMENTS

Referring initially to FIG. 1, a cryoablation system in accordance with the present invention is shown and generally designated 10. As shown, the system 10 includes a catheter tube 12 that extends from a proximal end 14 to a distal end 16. As further shown, the catheter tube 12 is formed with a lumen 18 that extends between the proximal end 14 and the distal end 16 of the catheter tube 12.

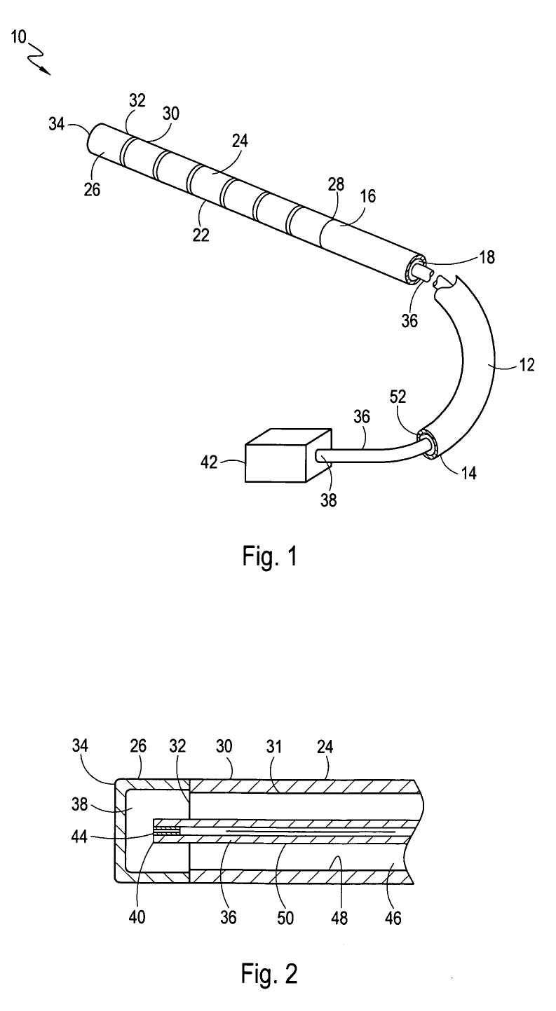

Continuing with FIG. 1, it can be seen that the system 10 also includes a cryotip 22 that includes a contact segment 24 and a cryo-element 26, both of which are made of thermally conductive materials. FIG. 1 shows that the contact segment 24 extends from a proximal end 28 to a distal end 30, and as shown in FIG. 2 is formed with a lumen 31. It can be further seen that the proximal end 28 of the contact segment 24 is directly attached to the distal end 16 of the catheter tube 12. Structurally, the proximal end 32 of the cryo-element 26 is directly attached to the distal end 30 of the contact segment 24, establishing a thermally conductive interface which allows heat to flow somewhat easily between the contact segment 24 and cryo-element 26. As best seen in FIG. 2, the cryo-element 26 has an open proximal end 32, a closed distal end 34 and surrounds an expansion chamber 35.

Cross-referencing FIGS. 1 and 2, it can the seen that the system 10 also includes a supply tube 36 that extends from a proximal end 38 to a distal end 40. The proximal end 38 of the supply tube 36 is connected to a refrigerant supply unit 42. From the proximal end 38, the supply tube 36 passes through the lumen 18 of the catheter tube 12 and the lumen 31 of the contact segment 24 and projects slightly into the expansion chamber 35. A restriction 44 is positioned in the supply tube 36 at the distal end 40 to restrict the flow of refrigerant. A refrigerant return line 46 is established having a first portion that extends between the inner surface 48 of the contact segment 24 and the outer surface 50 of the supply tube 36 and a second portion that extends between the inner surface 52 of the catheter tube 12 and the outer surface 50 of the supply tube 36.

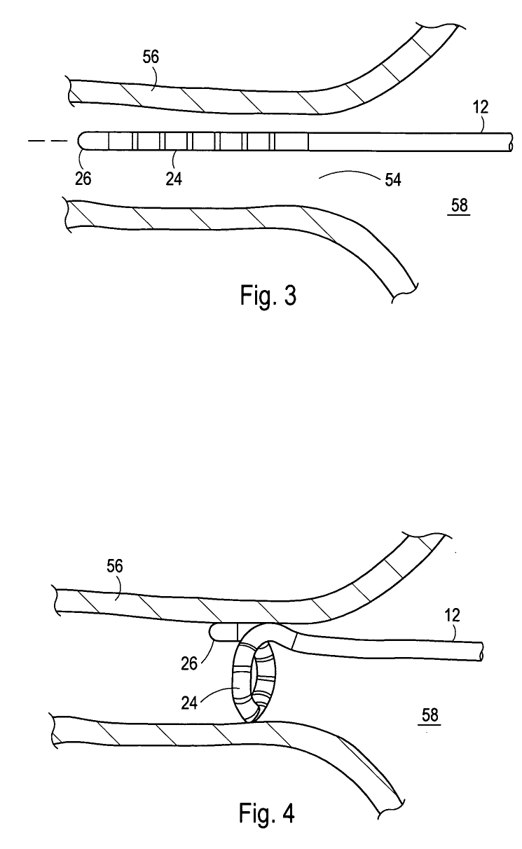

For the system 10, the contact segment 24 is typically made of a thermally conductive material having two-way shape memory such as an alloy of nickel and titanium (e.g. Nitinol). As shown in FIGS. 3 and 4, the contact segment 24 is transformable in response to a temperature decrease from a first, relatively straight configuration (shown in FIG. 3) to a second, substantially coiled configuration (shown in FIG. 4). In addition, the contact segment 24 is transformable from the coiled configuration (FIG. 3) back to the relatively straight configuration (FIG. 3) in response to a temperature increase. In the relatively straight configuration (FIG. 3), the contact segment 24 can be somewhat easily passed through the vasculature to (and from) a treatment site. On the other hand, when the contact segment 24 is in the coiled configuration (FIG. 4), the contact segment 24 can be cooled to a cryogenic temperature to cryoablate a circumferential band of tissue in a one-step process.

In greater detail, the contact segment 24 is made of a thermally-conductive, shape memory material that has been formed having a preset, relatively straight shape (FIG. 3) at a first, relatively high temperature (T₁), and having a preset, substantially coiled shape (FIG. 4) at a second, relatively low temperature (T₂). Moreover, as shown in FIG. 4, the contact segment 24 in the coiled configuration has a sufficient coil diameter to establish contact with the circumferential band of tissue that is to be cryoablated.

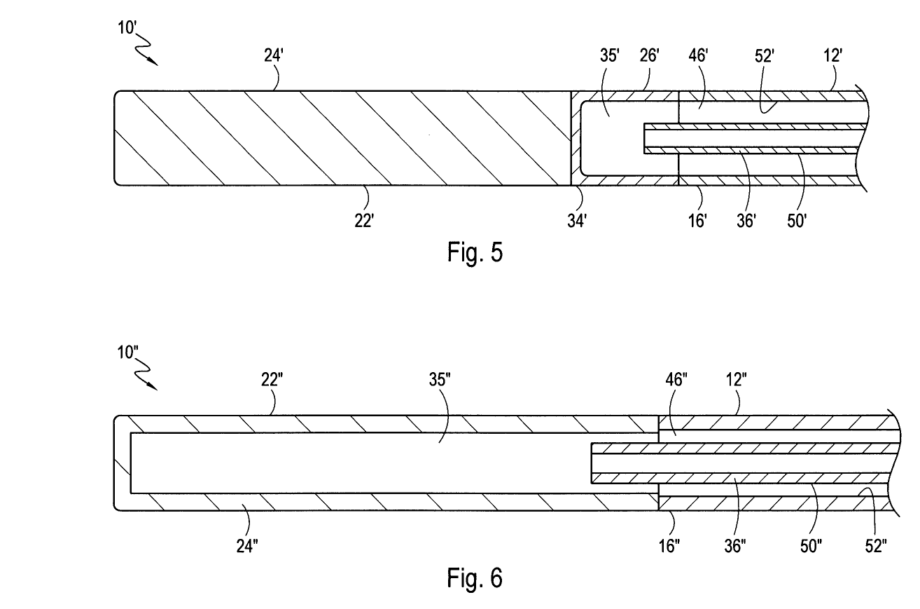

FIG. 5 shows an alternate embodiment of a cryoablation system 10′ having a cryo-element 26′ that is attached to the distal end 16′ of a catheter tube 12′. As further shown for this embodiment, a contact segment 24′ which can be a hollow tube or a solid (as shown) is attached to the distal end 34′ of the cryo-element 26′. Also shown in FIG. 5, a supply tube 36′ extends partially into the expansion chamber 35′ and establishes a return line 46′ between the inner surface 52′ of the catheter tube 12′ and the outer surface 50′ of the supply tube 36′.

FIG. 6 shows yet another embodiment of a cryoablation system 10″ having a cryotip 22″ that includes a contact segment 24″ that surrounds and establishes an expansion chamber 35″. For this embodiment, the contact segment 24″ is attached directly to the distal end 16″ of a catheter tube 12″. Also shown in FIG. 6, a supply tube 36″ extends partially into the expansion chamber 35″ and establishes a return line 46″ between the inner surface 52″ of the catheter tube 12″ and the outer surface 50″ of the supply tube 36″.

### Operation

The operation of the system 10 (and by analogy, systems 10′ and 10″) can best be appreciated with reference to FIGS. 3 and 4 which show a treatment site at the ostium 54 of a pulmonary vein 56 where the pulmonary vein 56 connects to the left atrium 58. Referring to FIG. 3, the contact segment 24 is initially set to a temperature that is at or above the first temperature (T₁) to thereby configure the contact segment 24 into the straight configuration. Preferably, the first temperature (T₁) is in the range of minus 55 degrees Celsius to 37 degrees Celsius. While in the straight configuration, the contact segment 24 has the required flexibility that allows it to be advanced through, and positioned in, the vasculature of a patient. Specifically, the catheter tube 12 is used to advance the contact segment 24 to the treatment site. At the treatment site, the cryotip 22 is positioned near the target tissue to be cryoablated.

With cross-reference now to FIGS. 1 and 4, it is to be appreciated that once the contact segment 24 is positioned at the treatment site, a fluid refrigerant from the refrigerant supply unit 42 is transferred through the supply tube 36 and into the expansion chamber 35 (FIG. 2) of the cryo-element 26. Inside the expansion chamber 35, the fluid undergoes endothermic expansion to absorb heat from the cryo-element 26. Typically, a fluid refrigerant is used that transitions from a liquid state to a gaseous state as it expands into the expansion chamber 35. Heat absorbed by the refrigerant during this phase transition (i.e. latent heat) cools the cryo-element 26, which in turn cools the contact segment 24. After expansion, the gaseous fluid refrigerant can pass through the return line 46 (FIG. 2) and exit the patient at the proximal end 14 of the catheter tube 12.

The flow of fluid refrigerant is continued until the cryo-element 26 and contact segment 24 are cooled and they are both substantially at the second temperature (T₂), which is typically about minus 85 degrees Celsius. As a consequence of the contact segment 24 being cooled to the second temperature (T₂), the contact segment 24 transforms from the straight configuration (FIG. 3) to the coiled configuration (FIG. 4) and contacts (and cryoablates) a circumferential band of tissue surrounding the ostium 54.

After the target tissue has been cryoablated, the contact segment 24 can be warmed (e.g. to the first temperature (T₁)) to transform the contact segment 24 into the straight configuration (FIG. 3). For example, the contact segment 24 can passively absorb ambient heat at the treatment site to warm the contact segment 24. It will be appreciated, however, that the contact segment 24 can also be warmed by any other devices or methods known to those skilled in the pertinent art. Once straight, the contact segment 24 can then be withdrawn from the treatment site and removed from the patient.

While the particular cryoablation catheter system and method as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

1. A system for cryoablating tissue in the vasculature of a patient which comprises:
a catheter tube having a proximal end and a distal end;
an elongated cryotip attached to said distal end of said catheter tube, said cryotip being transformable in response to a temperature change between a first configuration, wherein at a first temperature (T₁) said cryotip is substantially straight, and a second configuration, wherein at a second temperature (T₂) said cryotip is curved; and
a means for cooling said cryotip from said first temperature (T₁) to said second temperature (T₂) to reshape said cryotip into contact with the tissue for cryoablation of the tissue at said second temperature (T₂).

2. A system as recited in claim 1 wherein at least a portion of said cryotip is made of a shape memory material.

3. A system as recited in claim 2 wherein the shape memory material is a nickel-titanium alloy.

4. A system as recited in claim 2 wherein the shape memory material is a two-way shape memory material.

5. A system as recited in claim 1 wherein said first temperature (T₁) is in the range of approximately minus 55 degrees Celsius to approximately 37 degrees Celsius.

6. A system as recited in claim 1 wherein said second temperature (T₂) is approximately minus 85 degrees Celsius.

7. A system as recited in claim 1 wherein said cryotip is dimensioned to contact tissue around the periphery of the ostium of a pulmonary vein when said cryotip is in said second configuration.

8. A system as recited in claim 1 wherein said catheter tube is formed with a supply lumen, said cryotip comprises a contact segment and a cryo-element having an expansion chamber in fluid communication with said supply lumen, and wherein said cooling means comprises a fluid source in fluid communication with said supply lumen for introducing a fluid refrigerant from said fluid source through said supply lumen and into said expansion chamber to cool said cryotip.

9. A system as recited in claim 8 wherein the fluid refrigerant is nitrous oxide.

10. A system as recited in claim 8 wherein said catheter tube is formed with a return lumen in fluid communication with said expansion chamber for removing the refrigerant fluid from said expansion chamber through said return lumen.

11. A system for cryoablating tissue at a predetermined site in the vasculature of a patient which comprises:
an elongated contact segment made of a shape memory material, said contact segment being transformable in response to a temperature change between a first configuration, wherein at a first temperature (T₁) said contact segment is substantially straight, and a second configuration, wherein at a second temperature (T₂) said contact segment is curved;
a means for advancing said contact segment to the predetermined site with said contact segment in said first configuration;
and
a means for transforming said contact segment at the predetermined site into said second configuration to contact the tissue for cryoablation of the tissue at said second temperature (T₂).

12. A system as recited in claim 11 wherein said contact segment is dimensioned to contact tissue around the periphery of the ostium of a pulmonary artery when said contact segment is in the second configuration.

13. A system as recited in claim 11 wherein said means for advancing said contact segment comprises a catheter tube having a proximal end and a distal end, with said contact segment attached to said distal end of said catheter tube to advance said cryotip to the predetermined site.

14. A system as recited in claim 11 wherein said transforming means comprises a cryo-element formed with an expansion chamber and a fluid source for introducing a refrigerant fluid from said fluid source into said expansion chamber to cool said contact segment.

15. A system as recited in claim 11 wherein said shape memory material is a nickel titanium alloy.

16. A method for cryoablating tissue at a predetermined site in the vasculature of a patient which comprises the steps of:
providing an elongated contact segment made of a shape memory material, said contact segment being transformable in response to a temperature change between a first configuration, wherein at a first temperature (T₁) said contact segment is substantially straight, and a second configuration, wherein at a second temperature (T₂) said contact segment is curved;
advancing said contact segment to the predetermined site with said contact segment substantially at said first temperature (T₁);
cooling said contact segment to said second temperature (T₂) at the predetermined site to transform said contact segment into said second configuration to contact the tissue; and
cryoablating the tissue.

17. A method as recited in claim 16 wherein the shape memory material is a nickel titanium alloy.

18. A method as recited in claim 16 wherein said cooling step is accomplished by:
attaching a cryo-element formed with an expansion chamber to said contact segment; and
expanding a fluid refrigerant in said expansion chamber.

19. A method as recited in claim 16 wherein the tissue is tissue around the periphery of the ostium of a pulmonary artery.

20. A method as recited in claim 16 wherein the first temperature (T₁) is in the range of approximately minus 55 degrees Celsius to approximately 37 degrees Celsius, and wherein the second temperature (T₂) is below approximately minus 85 degrees Celsius.
